(19) United States
(12) Patent Application Publication
(10) Pub. No.: US 2011/0137094 A1
(43) Pub. Date: Jun. 9, 2011

(54) PROCESS FOR PRODUCING ETHYLIDENE NORBORNENE

(76) Inventors: Enrico Ammannati, Vernio (IT); Sergio Foschi, Ferrara (IT); Francesco Guarna, Prato (IT)

(21) Appl. No.: 12/737,754

(22) PCT Filed: Aug. 10, 2009
(86) PCT No.: PCT/EP2009/060324

§ 371 Date: Feb. 14, 2011

(30) Foreign Application Priority Data
Aug. 19, 2008 (IT) MI2008A001523

(51) Int. Cl. C07C 2/00

(52) U.S. Cl. 585/315

(57) ABSTRACT

Process for producing ethylidenorbornene (ENB) comprising a stage of thermal cracking of DCPD to CPD carried out in an inert fluid to which it is fed a stream of DCPD comprising virgin DCPD from cracking containing up to 10% wt of tetrahydroindene (THI) and recycled DCPD containing THI coming from the subsequent stage of formation of vinylnorbornene. The contact time of DCPD with the heat transfer fluid is of few seconds and it is sufficient to achieve a conversion of said DCPD ≧95%, with little formation of oligomers. THI is then separated from the heat transfer fluid substantially free from DCPD and enriched in THI to a fractionation column.

### CROSS-REFERENCE TO RELATED APPLICATIONS

The present patent application is a nationalization of International Application No. PCT/EP2009/060324, filed Aug. 10, 2009, published in English, which is based on, and claims priority from, Italian Patent Application No. MI 2008A001523, filed Aug. 19, 2008, both of which are incorporated herein by reference in their entireties.

### DESCRIPTION

The present invention relates to a process for producing ethylidene norbornene. Ethylidene norbornene (5-vinyl-2-norbornene, or ENB) is a useful chemical intermediate, particularly as termonomer for producing ethylene-propylene-diene rubber (EPDM). It is generally obtained by a multi-steps process comprising: a) thermal cracking of dicyclopentadiene (DCPD) to cyclopentadiene (CPD); b) Diels-Alder cycloaddition reaction of 1,3-butadiene with cyclopentadiene to form vinylnorbornene (VNB); c) catalytic isomerisation of vinylnorbornene to ethylidenenorbornene (ENB). Such reaction steps are associated to steps of separation and purification of the reaction products and/or other components of the system.

The present invention is particularly concerned with the stage of thermal cracking of dicyclopentadiene to cyclopentadiene, described by the following reaction scheme:

DCPD→2 CPD

The starting dicyclopentadiene is a low melting solid derivative obtained from cracking and distillation processes. It may contain variable amounts of by-products or impurities, mainly from 3 to 15% by weight of tetrahydroindene (THI).

Numerous processes for thermal cracking of DCPD to CPD are known.

U.S. Pat. No. 5,877,366 discloses a process for cracking DCPD to CPD in which the starting DCPD has a purity of 97%. Cracking of the diene dimer is carried out in a stirred reactor containing an inert heat transfer fluid consisting of, for example, molten diphenylether, at a temperature between 230 and 260° C. Given the high purity of the starting DCPD, this patent does not take into consideration the problem represented by the purification of the heat transfer fluid from by-products or impurities that accumulate in said fluid in a continuous process.

U.S. Pat. No. 5,565,069 discloses a process for producing ENB comprising an initial step in which DCPD is cracked to CPD. This patent uses a DCPD with a significant amount of THI, between 5/100 to 80/100, which has a boiling point of 160° C., and such THI partially reacts with CPD formed from the cracking of diene dimer, thereby decreasing the yield of the process. To prevent such decrease of the yield of desired end product, the process disclosed in this patent teaches to distil the reaction mixture into a main CPD (up to 70%) containing fraction and a DCPD+THI containing fraction, followed by multiple distillations until distilling this latter fraction to separate THI from DCPD. Since the boiling point of DCPD is of 170° C. and the boiling point of THI is of 160° C., a separation of DCPD from THI by distillation in a continuous process is difficult, both for the very close boiling points and for the close proximity of such boiling points to the minimum cracking temperature of DCPD.

Moreover, the process disclosed in the cited patent document requires a reaction time for the thermal decomposition (cracking) in liquid phase of DCPD to CPD within the range of 10-120 minutes, preferably 10-60 minutes. The patent teaches also that if the reaction time of the thermal decomposition is shorter than 10 minutes, the decomposition rate of DCPD is low. Due to the high reactivity of the formed CPD, however, it appears that dimerization reactions and/or reactions with THI and/or oligomerization reactions are likely to occur if the formed CPD is maintained in the reaction mixtures for the periods of time disclosed in the cited patent. It is not true that the contact time has to be 10 min. The contact time right are seconds,

Especially at 215-235° C. Too much time contact lowers the yields of CPD, whatever DCPD is rich in TIE, and CPD reacts with THI and DCPD.

U.S. Pat. No. 4,777,309 discloses a continuous process for producing 5-vinyl-2-norbornene by the Dield-Alder reaction of CPD with butadiene, or a mixture of CPD and DCPD and butadiene. The process does not disclose the production of CPD by cracking DCPD or DCPD+THI mixtures and does not mention any critical aspects in using CPD alone vs. mixtures of CPD and DCPD. The same remarks apply to the process disclosed in U.S. Pat. No. 4,538,013.

The processes described in the patents above have the drawback of requiring use of a substantially pure starting DCPD, or to carry out a purification of the THI accumulated over the time by distillation, which involves the drawbacks discussed above. Also, with respect to U.S. Pat. No. 5,565,069, the process described therein has the drawback of requiring a rather long reaction time for the thermal decomposition of DCPD to CPD, which is likely to cause undesired reactions of the thus formed CPD, particularly reactions to form oligomers.

An object of the present invention is thus that of providing a process for producing ethylidene norbornene of the type generally described in U.S. Pat. No. 5,565,069, but in which the first reaction stage of thermal cracking of dicyclopentadiene to cyclopentadiene can be carried out as a continuous and commercially effective process that allows to obtain cyclopentadiene in high yield.

Another object of the present invention is to provide a process for producing ethylidene norbornene in which the first reaction stage of thermal cracking of dicyclopentadiene to cyclopentadiene is carried by minimizing undesired reactions of the as-formed cyclopentadiene.

A further object of the present invention is to provide a process for producing ethylidene norbornene in which the liquid reaction mixture from thermal cracking of dicyclopentadiene to cyclopentadiene is purified in a simple and effective way. Therefore the present invention relates to a process for producing ethylidene norbornene comprising the steps of:
a) Feeding DCPD containing THI to a first reactor for the thermal cracking of DCPD to CPD carried out in an inert heat transfer fluid having a boiling point >230° C., said thermal cracking being carried out at a temperature below the boiling point of said heat transfer fluid and comprised between 200° C. and 300° C.,
b) Feeding said CPD produced in said step a) to a second reactor in which said CPD is made to react with 1,3-butadiene to form vinylnorbornene (VNB);
c) Purifying and separating VNB of step b) from DCPD+THI
d) Feeding said VNB produced in said step c) to a third reactor in which a catalytic isomerisation of VNB to ethylidene norbornene (ENB) is carried out;
e) collecting said ENB;
   wherein said step a) is characterized in that:
i. said DCPD fed to said step a) comprises virgin DCPD from cracking containing up to 10% by weight of tetrahydroindene (THI) and recycled DCPD containing tetrahydroindene (THI) coming from said step b) in which vinylnorbornene is formed;
ii. said DCPD containing said THI is fed into said heat transfer fluid and is in contact with same for a time of less than 1 minute;
iii. the formed CPD vaporizes to the gas-phase established above said liquid phase and is continuously removed from said first reactor as it vaporizes;
iv. a part of said heat transfer fluid substantially free from DCPD and enriched of THI is continuously fed to a fractionation column in which THI is separated from said heat transfer fluid, said THI being collected at the head of said column and said heat transfer fluid being collected at the bottom of said column;
v. said heat transfer fluid purified in said step iv) is recycled to said first reactor of said step a).

According to another aspect of the invention, thermal cracking of DCPD of step a) is carried out in the presence of a polymerization inhibitor, such as tetramethylpiperidine, preferably its derivative as 4-oxo-TEMPO (4-oxo-2,2,6,6-tetramethylpiperidine-N-oxide or ter-butyl hydroquinone (TBHQ), or mixtures thereof. Such polymerization inhibitor is to present at a concentration by weight of from 500 to 5,000 ppm, preferably from 1,000 to 4,000 ppm, more preferably from 1,500 to 2,500 ppm.

The heat transfer fluid has preferably a boiling point of from 230 to 350° C., and is selected preferably from diphenylether, diphenylmethane, decalin, Dowtherm® oil (a mixture of diaryl and triaryl ethers). More preferably the heat transfer fluid is diphenylether, which has a boiling point of 258-260° C.

The step of thermal cracking of dicyclopentadiene to cyclopentadiene according to the invention allows to use as starting material a virgin DCPD from oil cracking, either pure or with a THI content of about 10% by weight, mixed with DCPD recycled from stage b), which can contain up to 15% by weight of THI. The flow rates of virgin DCPD and recycled DCPD are such as to maintain the level of THI in the heat transfer fluid below 2%, to minimize the formation of oligomers and co-dimers.

It has been found that in the process of the present invention the cracking of DCPD to CPD, at a temperature of from 200° C. to 300° C., occurs in less than 1 minutes. At such temperature the conversion of DCPD is thus of >94%. The very high conversion of DCPD achieved in a very low residence time has the favourable consequence that the heat transfer fluid withdrawn from the cracking reactor is substantially free from DCPD. This allows to carry out a simple and effective purification of the heat transfer fluid since—differently from known processes—the heat transfer fluid does not contain compounds with very close boiling points such as DCPD and THI, but it contains only THI, and its oligomerization products whose boiling point of 160° C. is much lower than the boiling point of the heat transfer fluid.

With respect to CPD, whose boiling point is of 41° C., it has been found that it vaporizes quickly, almost instantaneously as formed in the conditions used in the process. This means that CPD—as formed by cracking of DCPD in the liquid phase—transfers to the gas-phase established above the liquid phase in the cracking reactor. According to an aspect of the invention, the gaseous CPD as formed and as-vaporized is continuously removed from the gas-phase, so that undesired reactions of CPD are avoided or minimized. This is an important aspect of the process according to the present invention, since the high reactivity and low stability of CPD at the temperature established in the cracking reactor would cause parasite reactions to DCPD, or with THI to form oligomers. Use of a polymerization inhibitor has proven to be effective in contributing to avoid or minimize such reactions. According to another aspect of the process of the invention, continuous removal of gaseous CPD from the cracking reactor as it vaporizes is carried in a selective way, namely only CPD is removed while other vapour-phase components are left in the reactor. This is achieved by outlet means in the reactor provided with heat transfer means set at a temperature that maintains CPD in the gas-phase but causes higher boiling components to condense. For example such outlet means comprises a heat exchanger in the upper part of the cracking reactor, equipped with a reflux condenser adjusted at a temperature equal to or slightly below the boiling point of CPD, so that CPD is selectively allowed to exit the reactor but any other components with a boiling point higher than that of CPD are condensed and refluxed back into the reactor, so that they cannot leave the reactor. The temperature for selective withdrawal of CPD is from 37 to 45 C, preferably 39-41, depending on heat exchanger design. According to this feature, CPD is thus condensed outside the cracking reactor and withdrawn as a condensed liquid.

CPD produced in the stage a) is strictly kept at a low temperature before it is fed to stage b).

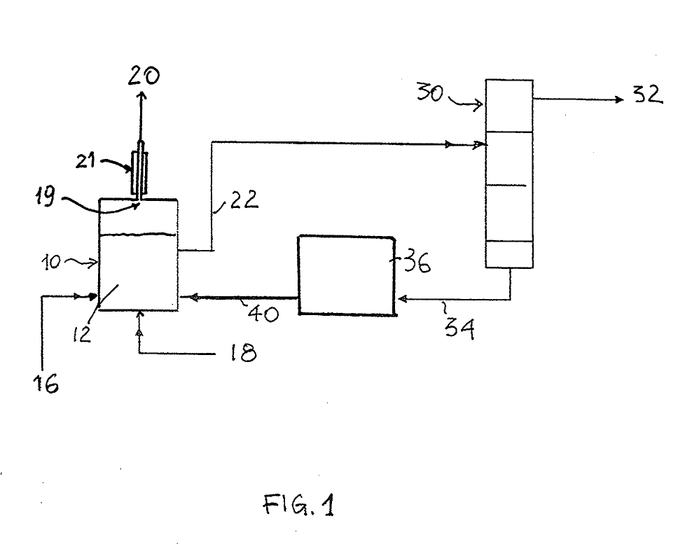

In the following description step a) of thermal cracking of DCPD according to the invention is described by referring to a simplified scheme shown in FIG. 1.

In such embodiment 10 is a reactor provided with heating means and stirring means, not shown. Reactor 10 contains a heat transfer fluid 12 comprising molten diphenyl ether kept at a temperature of 215° C. By means of a line 16 virgin DCPD with a THI content of 10% by wt. is fed to the reactor, and recycled DCPD with a THI content of 14% by wt. is fed to reactor 10 by line 18.

The amount of diphenylether and the flow rates of DCPD fed are such that the THI content in the diphenylether is never above 2% by weight.

The contact time between DCPD and diphenylether at 215° C. is of 10-30 seconds, sufficient to achieve an almost complete cracking of DCPD to CPD, with a conversion of ≧94%, as said above. Due to its low boiling point, this latter vaporizes and transfers immediately as formed to the gas-phase above the level of liquid diphenyl ether. The gaseous CPD is thus removed from reactor 10 via an outlet port 19 and line 20 and sent to a parallel condensation stage. Selective removal of CPD is achieved by connecting outlet port 19 to a reflux condenser 21 in which cooling means are set to maintain the temperature of the outlet CPD at 40° C. higher boiling components are condensed and refluxed into the reactor.

Preferably starting DCPD is pre-heated at a temperature of about 140-160° C. before it is fed to reactor 10.

A part of diphenylether is continuously withdrawn via line 22 and sent to a fractionation column 30. Since the cracking of DCPD to CPD is almost immediate and complete, namely the conversion of DCPD is ≧94%, the diphenylether withdrawn via line 22 is substantially free from DCPD. The diphenylether enriched with THI is then fed to a column 30, where fractionation is carried out by distillation under vacuum. THI is collected at the top of the column and removed via line 32. Purified diphenylether at a temperature of 190-200° C. is withdrawn at the bottom of the fractionation column via line 34 and sent to a storage tank 36, from which it is recycled to reactor 10 via line 40, at a desired flow rate. The temperature of the diphenylether withdrawn at the bottom of the fractionation column 30 is close to temperature of thermal cracking of DCPD.

Two runs for thermal cracking of DCPD to CPD were carried out by using a laboratory scale reactor to which DCPD+THI were fed via a single line, at the following conditions:

### Run 1

Reactor for thermal cracking: Laboratory scale cylindrical reactor, height 29 cm, diameter 16 cm; capacity 5.83 litres, immersed in a silicon oil bath heated by 2 electric resistances of 500 watt each. The reactor had a CPD outlet port equipped with heat exchanger and reflux condenser, as described in connection to reactor 10 above;

### Stirrer: Mechanical, 550 rpm

Amount of diphenylether: 500 g
Oligomerization inhibitor (TBHQ): 2500 ppm
T reactor: External 250-260° C.; Internal 215° C.
T_{CPD} withdrawn from top of reactor: 41° C.

A mixture of 445.9 g of DCPD and 44.1 g THI, amounting of 490 g, was fed dropwise into the cracking reactor above the level of liquid diphenylether, at a flow rate of 3 ml/min, for a time of 155 min. It was observed that CPD vaporizes immediately as formed and was continuously removed as condensed liquid.

At the end of the run 424 g of CPD with a purity of 99.04% were obtained, corresponding to 420 g of pure CPD. The balance was 0.527% THI and 0.4276% DCPD. The yield of the cracking reaction DCPD→2 CPD with respect to CPD was of 94.19% (420/445.9×100).

A mixture of 536 g of exhaust diphenylether was also obtained. The mixture had the following composition: diphenylether 90.36%, THI 6.13%, DCPD 0.37%, oligomers 3.14%. This mixture was sent to a fractionation column, as described above, at the bottom of which purified diphenylether was obtained.

### Run 2

A mixture of 278.5 g of DCPD and 27.5 g oh THI was fed to the same reactor and at the same conditions as above. The mixture was fed dropwise into the cracking reactor above the level of liquid diphenylether, at a flow rate of 3 ml/min, for a time of 80 min. It was observed that CPD vaporizes immediately as formed and was continuously removed as condensed liquid.

At the end of the run 264 g of CPD with a purity of 98.36% were obtained, corresponding to 259.67 g of pure CPD. The balance was 0.82% THI and 0.55% DCPD. The yield of the cracking reaction DCPD→2 CPD with respect to CPD was of 93.24.

A mixture of 534 g of exhaust diphenylether was also obtained having the following composition: diphenylether 94.9%, THI 2.01%, DCPD 0.50%, oligomers 2.59%. This mixture was sent to a fractionation column, as described above, at the bottom of which purified diphenylether was obtained.

As it is evident from the description and the examples above, thermal cracking carried out according to the process of the invention has the advantage of using streams of DCPD containing significant amounts of THI without causing fouling of the reactor or clogging of the fluid transport lines due to formation of oligomers. The acceptable level of THI present in the reactor is determined on the basis of the flow-rate of virgin DCPD and recycled DCPD, respectively, and on the basis of the flow-rate of diphenylether purified in the fractionation column and recycled to the reactor. The process allows to maximize the yield of CPD, and purity although the starting diene dimer used is not pure.

The CPD produced at stage a) described above is then fed to the subsequent reaction stages, as described above, which are not described in detail since they are known to the skilled in the art.

1. Process for the production of ethylidennorbornene (ENB) comprising the steps of:
a) feeding DCPD to a first reactor for the thermal cracking of DCPD to CPD, carried out in inert heat transfer fluid having a boiling temperature >230° C., said thermal cracking being carried out at a temperature lower than the boiling temperature of said heat transfer fluid and comprised between 200° C. and 300° C.
b) feeding said CPD produced in said step a) to a second reactor in which said CPD is made to react with 1,3-butadiene to form vinylnorbornene (VNB);
c) feeding said VNB produced in said step b) to a third reactor in which a catalytic isomerisation of VNB to ethyllidene norbornene (ENB) is carried out;
d) collecting said ENB;
wherein said step a) is characterized in that:
i. said DCPD fed to said step a) comprises virgin DCPD from cracking containing up to 10% wt. of tetrahydroindene (THI), and recycled DCPD containing tetrahydroindene (THI) recycled from said step b) of formation of vinylnorbornene;
ii. said DCPD containing said THI is fed to said heat transfer fluid and is in contact with same for a time of less than 1 minute;
iii. the formed CPD vaporizes to the gas-phase established above said liquid phase and is continuously removed from said first reactor;
iv. a part of said heat transfer fluid substantially free of DCPD and enriched of THI is continuously fed to a fractionation column, said THI being collected at the head of said column and said heat transfer fluid being collected at the bottom of said column;
v. said heat transfer fluid purified in step iv) is recycled to said first reactor of said step a).

2. Process according to claim 1, characterized in that said continuous removal of as-vaporized CPD from said cracking reactor is carried in a selective way by providing outlet means provided with heat transfer means set at a temperature that maintains CPD in the gas-phase but causes higher boiling components to condense.

3. Process according to claim 2, characterized in that said heat transfer means comprises a reflux condenser adjusted at a temperature equal to or slightly above the boiling point of CPD, whereby said as-vaporized CPD is selectively allowed to exit the reactor but any other components with a boiling point higher than that of CPD are condensed and refluxed into said reactor.

4. Process according to claim 2, characterized in that said heat transfer means are set at a temperature for selective withdrawal of CPD of from 37° C. to 45° C.

5. Process according to claim 2, characterized in that said heat exchanger are set at a temperature for selective withdrawal of CPD from 39 to 41° C.

6. Process according to claim 2, characterized in that said CPD is condensed outside said cracking reactor and withdrawn as a condensed liquid.

7. Process according to claim 1, characterized in that said step a) is carried out in the presence of a polymerization inhibitor.

8. Process according to claim 7, characterized in that said polymerization inhibitor is selected from a tetramethylpiperidine or ter-butyl hydroquinone or their mixtures.

9. Process according to claim 7, characterized in that said polymerization inhibitor comprises the radical designated as 4-oxo-TEMPO (4-oxo-2,2,6,6-tetramethylpiperidina-N-oxy).

10. Process according to claim 7, characterized in that said polymerization inhibitor is present at a concentration by weight comprised between 500 and 5000 ppm.

11. Process according to claim 7, characterized in that said polymerization inhibitor is present at a concentration by weight comprised between 1000 and 4000 ppm.

12. Process according to claim 7, characterized in that said polymerization inhibitor is present at a concentration by weight comprised between 1500 and 2500 ppm.

13. Process according to claim 1, characterized in that said heat transfer inert fluid has a boiling temperature comprised between 230° C. and 350° C.

14. Process according to claim 1, characterized in that said heat transfer inert fluid is diphenyl ether.

15. Process according to claim 1, characterized in that said recycled DCPD containing tetrahydroindene (THI) recycled from said step b) of formation of vinyl norbornene comprises an amount of THI up to 15% wt.
